(19) United States
(12) Patent Application Publication
(10) Pub. No.: US 2011/0301598 A1
(43) Pub. Date: Dec. 8, 2011

(54) Electrocautery Cutting Instrument with Elastic Wire Loop

(76) Inventors: Juan J. Arias, Hollywood, FL (US); Martin P. Michalewski, Deerfield Beach, FL (US); Elvis Garcia, Houston, TX (US)

(21) Appl. No.: 12/795,150

(22) Filed: Jun. 7, 2010

(51) Int. Cl. A61B 18/14

(52) U.S. Cl. 606/45

(57) ABSTRACT

An electrocautery cutting instrument includes a shaft having a first handle at one end and an elastic wire loop at the other. An outer tube is longitudinally displaceable over the shaft to collapse and permit expansion of the wire loop. The wire loop is sized for surrounding the uterus during a gynecological procedure. The wire loop has bends that bias the loop into a planar configuration when the wire loop is in an expanded configuration. The distal portion of the wire loop is integrally formed of the same flexible wire as the proximal portion of the wire loop, but has a different cross-sectional shape from the proximal portion.

### BACKGROUND OF THE INVENTION

1. Field of the Invention

This invention relates broadly to surgical instruments. More particularly, this invention relates to electrosurgical cutting instruments for use in obstetrics and gynecological procedures, including hysterectomy.

2. State of the Art

Electrosurgical instruments and techniques are widely used in surgical procedures because they generally reduce patient bleeding and trauma associated with cutting operations, as compared with mechanical cutting. In gynecological procedures it is known to use flexible loop electrocautery cutting instruments to cut and remove soft tissue. For example, U.S. Pub. No. 20090254082, discloses a flexible loop electrocautery cutting instrument having an expansible wire loop, a shaft extending proximally from the loop, and a handle at the proximal end of the shaft. An electrosurgical generator can be removably coupled to the wire loop via the shaft to cauterize tissue in contact with the wire loop. An outer tubular member is slidably displaceable over the shaft and wire loop to cause the wire loop to controllably move between collapsed and expanded configurations. In the collapsed configuration, the wire loop may be introduced through a small port opening into the patient's body. In the expanded configuration, the wire loop is sized for surrounding the tissue to be removed.

Such a flexible loop electrocautery cutting instrument can be used for various surgical procedures. For example, it can be used to remove fibroids. It is also known to use such an instrument having an appropriately loop of sufficient size to ensnare the uterus so that the uterus can be sectioned from the cervix in a laparoscopic total or supracervical hysterectomy (LSH).

The loops of such instruments are generally structured of thin wire not exceeding 0.025 inch in diameter. When the loop is of a relatively large size, e.g., exceeding six inches between its proximal and distal ends, such as required for performing a hysterectomy, maintaining directional control of the loop from the handle can be difficult. That is, there is a tendency for the loop to lack planar stability and flop, even if the loop is provided with a stiffening sheath over portions not required for cautery cutting. As a result, the time required to complete a procedure is longer than necessary, an additional instrument and port therefor to aid and guide the loop about the uterus is sometimes necessary. Further, where the loop instrument is used unaided, in view of the lack of stability and resultant lack of directional control, the surgical procedure becomes more complicated and risky.

### SUMMARY OF THE INVENTION

In accord with an embodiment of the invention, an electrocautery cutting instrument suitable for use gynecological surgical procedures is provided. The instrument includes a rigid element, such as a shaft, having a proximal end, a distal end, and defining a longitudinal axis. A first handle is fixed at the proximal end of the shaft and a flexible wire loop is coupled to the distal end of the shaft. The wire loop is elastically expansible into an expanded preferably substantially planar configuration in which the wire loop defines a length extending parallel to the longitudinal axis and a width transverse to the length. The length and width are sized for surrounding the uterus during a gynecological procedure such as a laparoscopic total or supracervical hysterectomy (LSH). An outer tube is provided over the shaft and has a proximal second handle. The outer tube is slidably displaceable over both the shaft and the wire loop to cause the wire loop to collapse when the outer tubular member is in a relatively distal position and to allow the wire loop to expand when the outer tubular member is in a relatively proximal position.

The wire loop has a sequence of bends at a proximal end, a distal end, and a intermediate location thereof. These bends bias the wire loop into a planar configuration when the wire loop is in an expanded configuration. In one embodiment, the wire loop defines first and second arms. The first arm has a proximal first end extending parallel to the longitudinal axis, an adjacent second portion bent at an angle laterally outward from the proximal first end by 45±5° to the longitudinal axis, an elongate third portion bent at an angle laterally inward relative to the second portion so as to be oriented 20±5° laterally outward relative to the longitudinal axis, an elongate fourth portion bent at an angle laterally inward relative to the third portion so as to be oriented 20±5° laterally inward relative to the longitudinal axis, with the joining distal tip bent at an angle relative to the fourth portion to extend transverse to the longitudinal axis. The second arm is preferably bent in a similar manner such that the wire loop is bilaterally symmetrical. An electrically insulative sheathing is provided over a proximal portion of the wire loop, but leaves the distal portion of the wire loop exposed.

The distal portion of the wire loop is integrally formed of the same flexible wire as the proximal portion of the wire loop. The proximal portion has a round first cross-sectional shape, whereas the distal portion has a second cross-sectional shape, preferably with no more than four sides, such that a relatively increased cross-sectional cutting area (relative to the first cross-sectional shape) is provided for contacting the tissue of the uterus as said wire loop is collapsed about the uterus. The second cross-sectional shape is preferably triangular or rectangular.

According to another aspect of the invention, a method of imparting both the cross-sectional shape to only a portion of the wire, as well as the method of bending the wire loop into a shape in accord with a preferred aspect of the invention is provided. Dies are provided through and between which the wire loop is positioned. Compression forces are applied to the dies to effect both the angular bending of the elastic loop and the change in cross-sectional shape at the distal end of the loop relative to a more proximal portion of the loop.

In use, the second handle is displaced from the first handle to cause the outer tube to collapse the wire loop. When the wire loop is in the collapsed position the distal end of the instrument may be introduced through a port and into the patient for purpose of the surgical procedure. The first and second handles are moved relative to each other to cause the outer tube to be longitudinally displaced so as to permit the loop to expand. The first handle is maneuvered to position the loop about target tissue, such as the uterus. The first and second handles are then manipulated to advance the outer tube so as to cinch the wire loop about the tissue, and the wire loop is energized with power from an electrosurgical generator to cauterize the tissue. The first handle is then retracted relative to the first second handle to fully collapse the wire loop into the outer tube, and the instrument is retracted from the patient.

Additional objects and advantages of the invention will become apparent to those skilled in the art upon reference to the detailed description taken in conjunction with the provided figures.

### BRIEF DESCRIPTION OF THE DRAWINGS

FIG. 1 is a perspective view of an electrocautery cutting instrument according to the invention.

FIG. 2 is a side elevation of the electrocautery cutting instrument of FIG. 1 shown with the handles positioned so that the wire loop is in a fully expanded configuration.

FIG. 3 is a side elevation of the electrocautery cutting instrument of FIG. 1 shown with handles positioned so that the wire loop is in a partially collapsed configuration.

FIG. 4 is a side elevation of the electrocautery cutting instrument of FIG. 1 shown with handles positioned so that the wire loop is fully collapsed.

FIG. 5 is a plan view of an embodiment of the wire loop of the electrocautery cutting instrument of FIG. 1.

FIG. 6 is a cross-section taken across line 6-6 in FIG. 5.

FIG. 7 is a broken top view of the distal end of the wire loop.

FIG. 8A is a first embodiment of cross-sectional shape of the distal end of the wire loop taken across line 8-8 in FIG. 7.

FIG. 8B is a second embodiment of cross-sectional shape of the distal end of the wire loop taken across line 8-8 in FIG. 7.

FIG. 8C is a third embodiment of cross-sectional shape of the distal end of the wire loop taken across line 8-8 in FIG. 7.

### DETAILED DESCRIPTION OF THE PREFERRED EMBODIMENTS

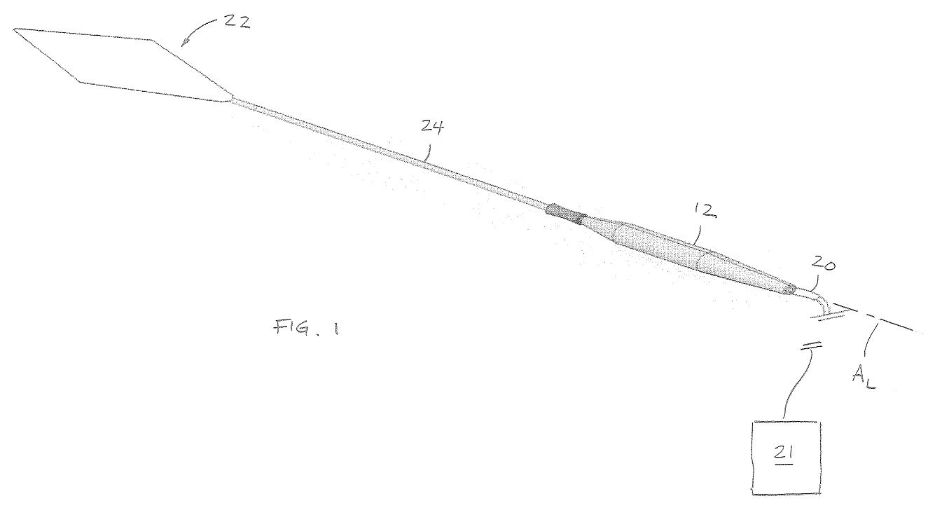

Turning now to FIG. 1, an electrocautery cutting instrument 10 suitable for use in gynecological surgical procedures is provided. The instrument 10 includes a first handle 12 having an elongate shaft 14 extending from the first handle. The first handle 12 is preferably made from a non-conductive material such as plastic. The shaft 14, seen best in FIG. 3, is preferably a rigid tubular element, preferably made from stainless steel. The shaft 14 has a proximal end 16, attached to the handle, a distal end 18, and defines a longitudinal axis A_{L}. A cautery connector 20 is coupled to the first handle 12 for connection to a standard electrosurgical generator 21. A flexible wire loop 22, described in detail below, is coupled to the distal end 18 of the shaft 14, preferably via spot weld, crimping or swaging. The wire loop 22 is electrically coupled to the cautery connector 20 through the shaft 14.

The wire loop 22 is elastically expansible and collapsible as described below. Most preferably, the wire loop is made of a superelastic alloy such as nickel titanium. In the expanded configuration (FIG. 2) the wire loop defines a length extending parallel to the longitudinal axis A_{L} and a distended width at a longitudinally central portion transverse to the length. The length and width are sized for surrounding the uterus during a gynecological procedure such as a laparoscopic total or supracervical hysterectomy (LSH). Preferred dimensions include a length of four to ten inch and a width of one to five inches. Such dimensions ensure that the loop is suitable for adult human hysterectomy while permitting the loop to be collapsed for introduction through a port not exceeding 5 mm.

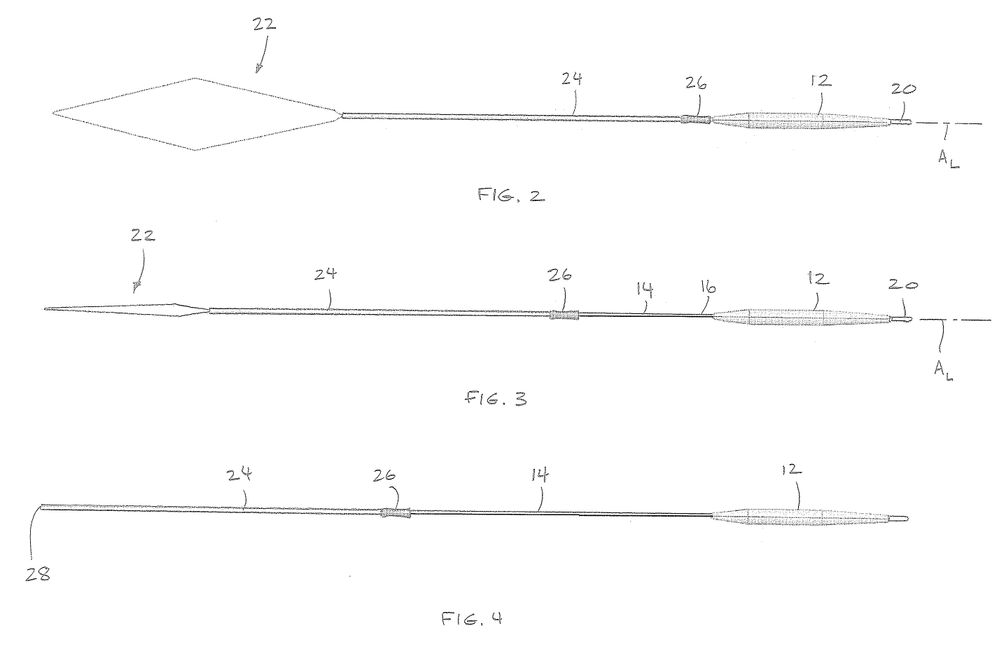

An outer tube 24 is provided over and slidably displaceable relative to the shaft 12 and the wire loop 22. The outer tube 24 is made of a non-conductive material, such as plastic, and includes a proximal second handle 26. As shown in FIG. 2, when the outer tube is in a relatively proximal position, second handle 26 is positioned adjacent the first handle 20, and the wire loop 22 is permitted to fully open into the expanded configuration. When the outer tube 24 is moved into a relatively distal position, it is advanced over the distal end 18 of the shaft 14 and over at least a portion of the wire loop 22 so as to cause cinching of the wire loop, as shown in FIG. 3. When the outer tube 24 is moved into a fully distal position, the wire loop is fully collapsed within the outer tube to permit the distal end of the instrument to be introduced or removed through a port and into the patient for purposes of the surgical procedure, as shown in FIG. 4. The distal end 28 of the outer tube is preferably sized to fully receive the distal end of the wire loop, including the distal tip 30, discussed below.

Turning now to FIG. 5, the wire loop 22 has a sequence of bends at a proximal end, a distal end, and a intermediate location thereof, such that in a preferred embodiment the wire loop assumes a diamond shape. These bends steady the wire loop to hold its form, and preferably bias the wire loop into a planar configuration, when the wire loop is in the expanded configuration and during when being placed over and around the uterus such that supplemental instrumentation is not required for the task.

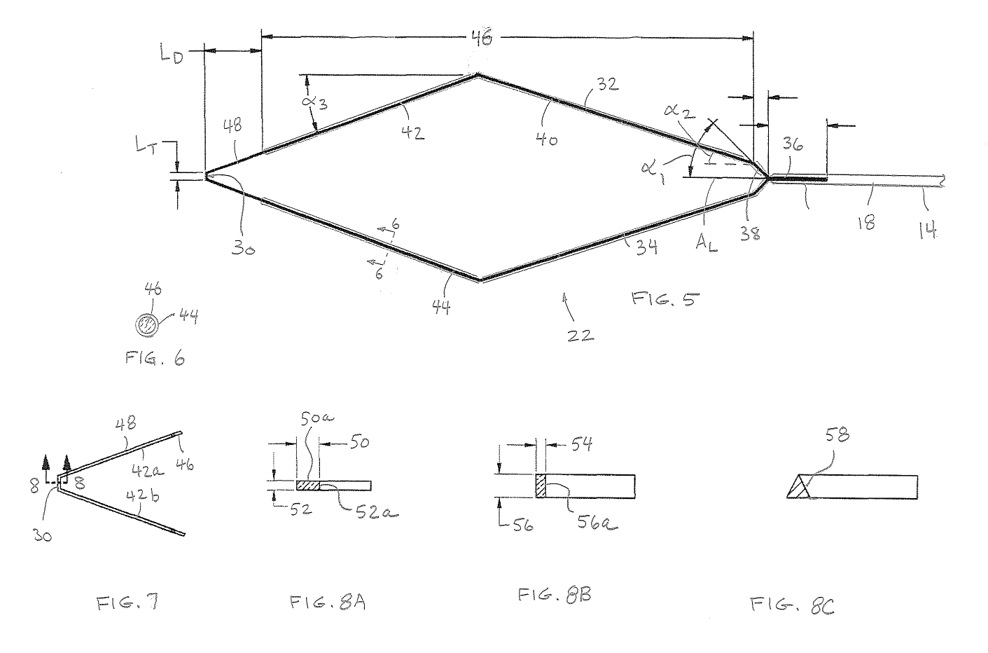

In one embodiment, the loop 22 and its bends are defined by the following. The wire loop 22 defines first and second arms 32, 34. The first arm 32 has a proximal first end 36 extending parallel to the longitudinal axis A_{L}. A second portion 38 extends from the first end 32 for approximately 0.25 inch of the loop length, and is bent at an angle laterally outward relative to the longitudinal axis A_{L} by an angle α₁, where α₁ is preferably 45±5°. An elongate third portion 40 extends from the second portion 38 and is bent at an angle α₂ laterally inward relative to the longitudinal axis A_{L}, where α₂ is preferably 20±5°. An elongate fourth portion 42 extends from the third portion 40 at an angle α₃ laterally inward relative to the longitudinal axis A_{L}, where α₃ is preferably 20±5°. The third and fourth portions 40, 42 are of substantially similar length (±10 percent). The distal tip 30 is bent at an angle relative to the fourth portion 42 to extend transverse to the longitudinal axis A_{L} and join the first and second arms 32, 34. As seen in FIG. 7, the fourth portions 42a, 42b of the first and second arms 32, 34 and the distal tip 30 together define a V-shape with a flattened apex. The distal tip 30 preferably has a length L_{T} transverse to the longitudinal axis A_{L} of 0.13 inch. The distal tip 30 is relatively incompressible about length L_{T}. The second arm 34 is preferably bent in substantially the same manner as the first arm 32 such that the wire loop 22 is bilaterally symmetrical. Referring to FIGS. 5 and 6, an electrically insulative sheath 44 is provided over a proximal portion 46 of the wire loop, but leaves the distal portion 48 of the loop, e.g., 12.5-25 percent of the length of the loop exposed or otherwise directly contactable against tissue. In one embodiment, the exposed distal portion 48 has a length L_{D} of 1 inch.

The exposed distal portion 48 of the wire loop is integrally formed unitary with and as an extension of the same flexible wire as the relatively proximal portions of the wire loop. The proximal portion 44 has a round first cross-sectional shape and the distal portion 48 has a second different cross-sectional shape. The phrase “cross-sectional shape” means a cross-section transverse to the longitudinal dimension of the wire; i.e., along line 6-6 in FIG. 5 for the first cross-sectional shape or along line 8-8 in FIG. 7 for the second cross-sectional shape. The first cross-sectional shape is preferably round or ovoid, as shown in FIG. 6. The second cross-sectional shape is polygonal, having discrete facets. The second cross-sectional shape preferably has no more than four facets. Referring to the embodiment of the second cross-sectional shape shown in FIG. 8A, the cross-sectional shape of the wire at the distal portion 48 has a rectangular shape with surfaces 50 of larger surface area in the plane of the loop and surfaces 52 of smaller surface area transverse to the plane of the loop. Thus, when the loop is cinched closed on tissue, the smaller surface 52a facing into the loop functions like a knife to cut through the tissue. The larger in-plane surface 50a allows for coagulation and/or improved hemostasis of vessels as the loop is moved through, and contacts and upper and lower bounding layers of the tissue. Referring to the embodiment of the second cross-sectional shape shown in FIG. 8B, the wire at the distal portion 48 has a rectangular shape with surfaces 54 of smaller surface area in the plane of the loop and surfaces 56 of larger surface area transverse to the plane of the loop. Thus, when the loop is cinched closed on tissue and a cautery voltage is applied to the loop, a larger cauterizing surface 56a (at the interior of the loop) is in contact with the tissue to cut through and subsequently cauterize the tissue. Referring to the embodiment of the second cross-sectional shape shown in FIG. 8C, the wire at the distal portion 48 has a triangular shape. The surfaces 58 are shown of equal size, but can be of relatively different sizes. The surfaces can be oriented to have planar, angular, or knife edge contact against the tissue. The shape of the distal end allows for greater cutting surface with improved tissue contact as the opposing tissue planes separate during the cutting process. In addition, the distal tip 30, transverse to the longitudinal axis A_{L}, facilitates pulling the cinched wire loop through tissue. The manner of imparting the different cross-sectional shapes to the wire loop is discussed below.

In use, the instrument 10 is prepared for insertion into a port by distally advancing the second handle 26 relative to the first handle 12 to cause the outer tube to collapse the wire loop 22 (FIG. 4). The distal end of the instrument 10 is inserted into the port and the second handle 26 is retracted relative to the first handle 12 to permit the loop 22 to expand (FIG. 2). The loop 22 is positioned around the uterus (not shown). The second handle 26 is then advanced to cinch to the loop 22 about the uterus (FIG. 3). The electrosurgical generator 21 (FIG. 1) is then activated, e.g., with a footswitch, to apply a cautery voltage and simultaneously the loop is pulled back through the tissue until the uterus is sectioned resulting in a clean, even, and non-charred cut that increases use of the cervical stump for pelvic support procedures for procedures such as laparoscopic sacral colpopexy. The loop, in view of the bends, offers a high degree of stability and resultant control. This decreases operating time significantly, up to 10 to 15 minutes on average. Further, by avoiding wandering of the loop in a less controlled instrument, safety is increased by ensuring amputation of the uterus at the desired level and prevention of the loop from moving lower to near previously secured uterine arteries.

According to another aspect of the invention, the method of imparting both the second cross-sectional shape to the distal portion of the wire, as well as the method of bending the wire loop into a shape in accord with a preferred aspect of the invention is provided. Compression dies are provided through and between which the wire loop is positioned. Compression forces are applied to the dies to effect both the angular kinking of the elastic loop and the change in cross-sectional shape at the distal end of the loop relative to a more proximal portion of the loop.

There have been described and illustrated herein embodiments of an electrocautery loop cutting instrument and methods of manufacturing the same. While particular embodiments of the invention have been described, it is not intended that the invention be limited thereto, as it is intended that the invention be as broad in scope as the art will allow and that the specification be read likewise. Thus, while particular materials have been disclosed, it will be appreciated that other materials can be used as well. In addition, while the electrocautery instrument is preferably a monopolar instrument, it will be understood that a second electrode can be provided to the instrument, e.g., on the outer tube, to construct a bipolar instrument. It will therefore be appreciated by those skilled in the art that yet other modifications could be made to the provided invention without deviating from its scope as claimed.

What is claimed is:

1. An electrocautery cutting instrument suitable for use gynecological surgical procedures, comprising:
a) a rigid elongate element having a proximal end, a distal end, and defining a longitudinal axis;
b) a handle fixed at said proximal end of the elongate element;
c) a flexible wire loop coupled to said distal end of said elongate element,
said wire loop elastically expansible into an expanded configuration in which said wire loop defines a length extending parallel to said longitudinal axis and a width transverse to said length, said length and width sized for surrounding the uterus,
said wire loop having a sequence of bends at a proximal end, a distal end, and a intermediate location thereof, wherein said bends bias said wire loop into a planar configuration when said wire loop is in said expanded configuration;
d) an insulative sheathing covering at least a proximal seventy-five percent of the length of the wire loop; and
e) an outer tube slidably displaceable over both the rigid element and the wire loop to cause the wire loop to collapse when the outer tubular member is in a relatively distal position and to allow the wire loop to expand when the outer tubular member is in a relatively proximal position.

2. An electrocautery cutting instrument according to claim 1, wherein:
said bends cause said wire loop to assume a diamond shape in said expanded configuration.

3. An electrocautery cutting instrument according to claim 1, wherein:
said wire loop includes a proximal portion and a distal cutting end integrally formed of the same flexible wire as said proximal portion, said proximal portion having a first cross-sectional shape, and said cutting end having a second cross-sectional shape different than said first cross-sectional shape.

4. An electrocautery cutting instrument according to claim 3, wherein:
said second cross-sectional shape includes facets.

5. An electrocautery cutting instrument according to claim 3, wherein:
said second cross-sectional shape is polygonal.

6. An electrocautery cutting instrument according to claim 3, wherein:
said second cross-sectional shape is triangular.

7. An electrocautery cutting instrument according to claim 3, wherein:
said second cross-sectional shape is rectangular.

8. An electrocautery cutting instrument according to claim 4, wherein:
said first cross-sectional shape is circular.

9. An electrocautery cutting instrument according to claim 3, wherein:
said second cross-sectional shape is formed in a compression die after said first cross-sectional shape is formed.

10. An electrocautery cutting instrument suitable for use gynecological surgical procedures, comprising:
a) a rigid elongate element having a proximal end, a distal end, and defining a longitudinal axis;
b) a first handle fixed at said proximal end of the elongate element;
c) a flexible wire loop coupled to said distal end of said elongate element,
said wire loop elastically expansible into an expanded configuration in which said wire loop defines a length extending parallel to said longitudinal axis and a width transverse to said length, said length and width sized for surrounding the uterus,
said wire loop defining first and second arms joined at a distal tip of the wire loop in a V-shape with a flattened apex, wherein the first arm has a proximal first end extending parallel to the longitudinal axis, an adjacent second portion bent at an angle laterally outward from the longitudinal axis by 45±5°, an elongate third portion bent at an angle laterally outward relative to longitudinal axis by 20±5°, an elongate fourth portion bent at an angle laterally inward relative to the longitudinal axis by 20±5°, with the joining distal tip bent at an angle relative to the fourth portion to extend transverse to the longitudinal axis,
wherein said bent angles between said first end and said second portion, said second portion and said third portion, and said third portion and said fourth portion of the first arm bias said wire loop into a planar configuration when said wire loop is in said expanded configuration; and
d) an outer tube slidably displaceable over both the elongate element and the wire loop to cause the wire loop to collapse when the outer tubular member is in a relatively distal position and to allow the wire loop to expand when the outer tubular member is in a relatively proximal position relative to said handle.

11. An electrocautery cutting instrument according to claim 10, further comprising:
an insulative sheathing covering a proximal portion of the wire loop; and
a cautery connector coupled to said first handle.

12. An electrocautery cutting instrument according to claim 10, wherein:
said first and second arms are bilaterally symmetrical.

13. An electrocautery cutting instrument according to claim 10, wherein:
the third and fourth portions of the first and second arms are of substantially similar length.

14. An electrocautery cutting instrument according to claim 10, wherein:
said distal tip extends in a dimension transverse to said longitudinal axis, and said outer tube has distal opening with inner diameter greater than length of distal tip in said transverse dimension.

15. An electrocautery cutting instrument according to claim 10, wherein:
said wire loop includes a proximal portion and a distal cutting end integrally formed of the same flexible wire as said proximal portion, said proximal portion having a first cross-sectional shape, and said cutting end having a second cross-sectional shape different than said first cross-sectional shape.

16. An electrocautery cutting instrument according to claim 15, wherein:
said second cross-sectional shape includes facets.

17. An electrocautery cutting instrument according to claim 15, wherein:
said second cross-sectional shape is polygonal.

18. An electrocautery cutting instrument according to claim 15, wherein:
said second cross-sectional shape is triangular.

19. An electrocautery cutting instrument according to claim 15, wherein:
said second cross-sectional shape is rectangular.

20. An electrocautery cutting instrument according to claim 16, wherein:
said first cross-sectional shape is circular.

21. An electrocautery cutting instrument according to claim 15, wherein:
said second cross-sectional shape is formed in a compression die after said first cross-sectional shape is formed.

22. An electrocautery cutting instrument suitable for use gynecological surgical procedures, comprising:
a) a rigid element having a proximal end, a distal end, and defining a longitudinal axis;
b) a first handle fixed at said proximal end of the rigid element;
c) a flexible wire loop coupled to said distal end of said rigid element,
said wire loop elastically expansible into an expanded configuration in which said wire loop defines a length extending parallel to said longitudinal axis and a width transverse to said length, said length and width sized for surrounding the uterus,
wherein said wire loop assumes a substantially planar configuration when said wire loop is in said expanded configuration,
said wire loop having a proximal portion and a distal cutting end integrally formed of the same flexible wire as said proximal portion, said proximal portion having a round first cross-sectional shape, and said cutting end having a second cross-sectional shape with no more than four sides, such that an increased cutting surface area is provided for contacting the uterus as said wire loop is collapsed about the uterus relative to said first cross-sectional shape;
d) an insulative sheathing covering a portion of the length of said wire loop; and
e) an outer tube slidably displaceable over both the rigid element and the wire loop to cause the wire loop to collapse when the outer tubular member is in a relatively distal position and to allow the wire loop to expand when the outer tubular member is in a relatively proximal position.

23. An electrocautery cutting instrument according to claim 22, wherein:
said second cross-sectional shape is rectangular.

24. An electrocautery cutting instrument according to claim 22, wherein:
said second cross-sectional shape is triangular.

25. An electrocautery cutting instrument according to claim 22, wherein:
said first cross-sectional shape is circular.

26. An electrocautery cutting instrument according to claim 22, wherein:
said second cross-sectional shape is formed in a compression die after said first cross-sectional shape is formed.
